(19) United States
(12) Patent Application Publication
(10) Pub. No.: US 2025/0102688 A1
(43) Pub. Date: Mar. 27, 2025

(54) IMAGING SYSTEM AND METHOD FOR RECORDING PROJECTION IMAGES

(71) Applicant: Siemens Healthineers AG, Forchheim (DE)

(72) Inventors: Stefan SCHAFFERT, Erlangen (DE); Thorsten ERGLER, Erlangen (DE)

(73) Assignee: Siemens Healthineers AG, Forchheim (DE)

(21) Appl. No.: 18/892,811

(22) Filed: Sep. 23, 2024

(30) Foreign Application Priority Data
Sep. 26, 2023 (EP) 23199724.8

(51) Int. Cl. G01T 1/24; G01T 1/29

(52) U.S. Cl. CPC G01T 1/242; G01T 1/2985; G01T 1/2992

(57) ABSTRACT

One or more example embodiments relates to an imaging system configured to record projection images, the imaging system comprising a detector unit; a radiation source, wherein the detector unit includes two detector elements arranged one behind the other, both of the two detector elements cover a same recording area, the two detector elements including a first detector element and a second detector element, and at least one of the radiation source or the detector unit is configured such that the second detector element is not saturated for sections of the recording area in which the first detector element is saturated during irradiation; and a data acquisition unit configured to record a first data set of the first detector element and to record a second data set of the second detector element such that first data set and the second data set are separable from one another.

### CROSS-REFERENCE TO RELATED APPLICATION(S)

The present application claims priority under 35 U.S.C. § 119 to European Patent Application No. 23199724.8, filed Sep. 26, 2023, the entire contents of which is incorporated herein by reference.

### FIELD

One or more example embodiments relates to an imaging system and a method for recording projection images.

### RELATED ART

In an X-ray image, which means a projection image recorded using X-rays, there is an area outside the subject area that is irradiated with primary radiation and scatter radiation from the subject. Because this area reduces as the lateral distance to the subject increases, the highest radiation intensities are usually found at the skin-air interface.

X-ray detectors are saturated if the intensity of the incident radiation (the primary radiation) exceeds the detector's dynamic range. Saturated pixel areas can no longer be used for image analysis or diagnosis. Saturated pixels are normally cropped as a result.

### SUMMARY

The dynamic range is limited by the minimum required sensitivity (digital resolution of the incident X-ray signal) and the maximum acceptable X-ray signal due to electronic restrictions (bit depth, maximum storage capacity). In contrast to radiography, the situation is intensified in mammography by the increased subject absorption used here of X-ray photon energies under 50 kVp.

One or more example embodiments specifies an imaging system and a method for recording projection images that will avoid the disadvantages of the related art.

### BRIEF DESCRIPTION OF THE DRAWINGS

One or more example embodiments is explained in detail below with reference to the attached figures based on exemplary embodiments. The different figures feature the same components with identical reference numbers. The figures are not generally to scale. In the drawings:

FIG. 1 shows an imaging system for recording projection images,

FIG. 2 shows data from imaging according to the prior art,

FIG. 3 shows data sets from imaging according to one or more example embodiments, and

FIG. 4 shows a method for recording projection images with an imaging system according to one or more example embodiments.

### DETAILED DESCRIPTION

The intensity in the skin-air interface area can change by several orders of magnitude. The detector's dynamic range is insufficient to capture all intensities for all observed subject thicknesses and densities in one image. Looking at the related art, it would be advantageous if this skin-air interface could be measured, as this would generate useful information, e.g., regarding the level of scatter radiation or incident X-ray intensity. The biggest challenge is that the skin-air interface is typically not imaged, i.e., cannot be clearly visualized, as the detector's saturation area intrudes into the actual image area.

Another key aspect is determining the total applied dose with high precision. This is not currently possible because of detector saturation. Major errors can arise as a result, for example, in the derived iodine accumulation in specific subject areas in contrast-enhanced dual-energy investigations.

A greater pixel storage capacity in the detector can expand the area of the incident X-ray signal for digitalization. However, this goes hand in hand with disadvantageous increased pixel noise.

Several images can also be used for the same subject, in order to divide the total clinical dose into smaller sections that can then be covered by the detector. This is also adversely affected by increased image noise with multiple interpretations, however, and the potential emergence of movement artifacts due to the longer overall capture time.

An imaging system according to one or more example embodiments is used to record projection images. The imaging system comprises a detector unit and a radiation source, wherein the detector unit has two detector elements arranged one behind the other, which both cover the same recording area. Additionally, the radiation source is configured and/or the detector unit is designed so that the second detector element is not saturated for sections of the recording area in which the first detector element is saturated during irradiation. Moreover, the imaging system includes a data acquisition unit that is designed to record a first data set from the first detector element and a second data set from the second detector element, so that the two data sets can be separated from one another.

A normal radiography system, fluoroscopy system or urology system, but also a system for tomography or tomosynthesis, can essentially be used as an imaging system. However, it must be fitted with a detector unit that has two detector elements arranged one behind the other. One or more example embodiments is ultimately advantageous for all applications in which projection images are recorded, regardless of whether these are the result or are used to reconstruct images.

The detector elements must be arranged so that an X-ray hits both detector elements simultaneously, i.e., both elements cover the same recording area. This would allow for correction here of the effect of a cone beam on differences in the imaging from the two detector elements. Such a correction is known in the prior art.

The data from the second detector element is required for the method to be applied advantageously. Therefore, this must not be saturated, at least in the areas whose data is used. Whether the second detector element becomes saturated depends on the one hand on the intensity of the primary radiation and, on the other, on the shielding effect of the first detector element and any additional absorbers. For this reason, the radiation source must be configured or the detector unit must be designed so that the second detector element is not saturated for sections of the recording area in which the first detector element is saturated during irradiation. As stated, this does not have to be all areas of the second detector element, but it should be the areas of a subject's interface to the air, as these are the most interesting.

Using the second detector element behind the first detector element thus facilitates the detection of X-rays that have passed through the first detector element. The radiation hitting the second detector element has a lower intensity due to absorption in the first detector element, so that the second detector element receives fewer, mainly high-energy photons. When the X-ray source is correctly configured, the second detector element can fully capture the pattern of the direct and scatter radiation and compensate for a saturated first detector element, without impairing the quality of the original image.

Suitable data acquisition units for the imaging system are certainly known. One or more example embodiments requires the data acquisition unit to be designed so that the first data set from the first detector element can be separated from the second data set of the second detector element. The data sets should be stored separately from one another or at least be labeled to facilitate clear assignment.

A method according to one or more example embodiments is used to record projection images with an imaging system as claimed in one of the previous claims. It includes the following steps:
irradiating a patient's body part with the radiation source, wherein both detector elements of the detector unit are irradiated through the body part,
reading both detector elements and forming a first data set by reading the first detector element and a second data set by reading the second data element,
generating a projection image from both data sets, wherein image values in the projection image, in which the first detector element was saturated during reading, are based on the second data set.

The recording of projection images using irradiation is sufficiently known. A feature of the method is that two detector elements are read here, and information is determined, e.g., supplemented, with the second data set, in areas in which the first detector element was saturated.

Further particularly advantageous embodiments and developments arise from the dependent claims and the description below, wherein the claims of one claim category can also be developed analogous to the claims and description sections of another claim category, and, in particular, individual features of different exemplary embodiments or variants can also be combined into new exemplary embodiments or variants.

In a preferred embodiment the radiation source is an X-ray source or particle radiation source. The imaging system is preferably a medical technology imaging system, e.g., a radiography system, especially for mammography.

A preferred imaging system includes a correction unit designed to correct at least one of the data sets. The correction unit is preferably designed for at least one of the following corrections: noise suppression, pixel registration, correction of cone beam enlargement, and/or spectral correction. Such correction units are known, especially in the form of software modules. Their use is advantageous for the method according to one or more example embodiments. Research time can be saved if the corrections can be applied where the first detector module was saturated and not to the first data set. It can also be advantageous if corrections are applied where the first data set is used to generate the projection image and not to the second data set, at least not if the second data set is not used for this.

A preferred imaging system includes an image generating unit that is designed to generate a projection image. More precisely, it is designed so that a first area of the projection image, especially a central area, is generated using data from the first data set. Data from the second data set can also be used, however, e.g., if this includes other spectral information. A second area of the projection image, especially a peripheral area, is generated using data from the second data set or using data from both data sets. Image data for which the first detector element was saturated is preferably reconstructed using data from the second data set, i.e., the second detector element. The data from the second data set can also just be used for this. This is scaled differently than the data of the first data set, however, as the second detector element experienced a different intensity. It is possible though to scale the data of the second data set in such a way that it matches the data of the first data set. Information from the first data set is also used for this, e.g., the progress in the area of the subject determines a scaling function for this data, and this scaling function is applied to all the data from the second data set (i.e., including where the first detector element was saturated).

The second detector element is preferably designed to receive higher energy radiation than the first detector element. This is advantageous, as radiation with a lower energy is predominantly completely absorbed in the first detector element. Spectral information is also produced, which can be used to generate the projection image.

The first detector element is preferably designed as an absorber. It should also be noted that all materials in a beam path can have an absorbing effect. This means that the detector element is designed as an absorber in addition to its function as a detector, e.g., a substrate that is thicker than normal is used for the detector material. In particular, the detection layer of the detector element, or a surface in or on a detection layer, is designed so that the second detector element does not become saturated with a predetermined radiation intensity for image recording.

The detector unit preferably includes an absorber plate between the first detector element and the second detector element, which covers, at least partially, the second detector element, wherein the absorber plate can preferably be removed or replaced. This enables precise shielding of the second detector element so that it does not become saturated.

According to a preferred embodiment variant of the method, a first area of the projection image is generated from the first data set (the information from the upper detector element). This first area shows the subject, e.g., the body part, and is in particular a central area of the projection image. It can also be generated with the help of the second data set, especially if this includes additional spectral information.

A second area is in particular a peripheral area around a central area and is generated using data from the second data set or a combination from both data sets. In the latter instance, the first data set is preferably used to adapt the second data set to the first data set.

The second area, especially a peripheral area, is preferably formed via a weighted convolution of both data sets or via scaling. This can be achieved in particular by using a convolution kernel or spectral filtering with a subsequent weighting of data from the second data set with the corresponding data from the first data set.

It is particularly preferable for a total dose over the entire image to be determined from the second data set and the first data set. This can be achieved, e.g., by first adapting the intensity measured by the second detector element in the second area, i.e., where the first detector element was saturated, to the first data set using the data from the first data set. The data in the second area would then reflect the case if the first detector element had not been saturated there. The progress of this data in the second area can now be used to draw conclusions about overall intensity and extrapolate these to the entire detector area. This can be done using formulas determined by test measurements without a subject or with the help of system correction tables.

According to a preferred embodiment variant of the method, the position and signal path on a surface of a recorded subject are extracted from the second data set. This is especially the position and signal path of a skin-air interface, e.g., on a breast. This information is then used to determine a corresponding signal path from the first data set. A lateral limit of a recorded subject, especially a skin-air interface, is then determined and/or corrected based on the second data set. It should be noted here that a saturation of the first detector element “blurs” information about the skin-air interface there.

At least one of the data sets is preferably corrected with regard to geometric effects related to dual-layer technology. A pixel registration of the first data set is preferably performed on the second data set or vice versa. Alternatively or additionally, a correction of the cone beam enlargement can also be performed. It should be noted here that the two detector elements are at different distances from the subject and the source and that a cone beam is typically used.

It is preferable for the recorded data sets to be spectrally corrected, especially on the basis of previously recorded calibration data. It is preferable to perform a beam hardening correction here. This allows for compensation of beam hardening effects due to the double-layer design of the detector unit. A priori knowledge can particularly be used for this. As the beam hardening of the detector unit is known, this can be used for the correction.

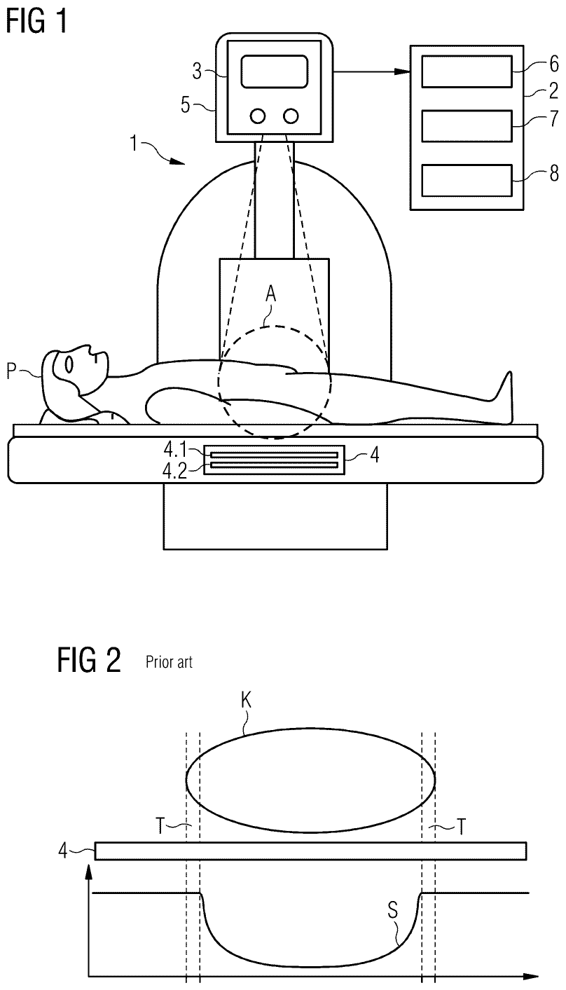

FIG. 1 shows a rough schematic of an imaging system 1 in the form of a radiography system 1 with a control device 2 for recording projection images. The radiography system 1 has a radiation source 3 in the customary manner, which here is an X-ray source, and radiographs a patient P during radiography imaging, so that the radiation hits a detector unit 4 positioned opposite the radiation source.

The detector unit 4 includes two detector elements 4.1, 4.2, arranged one behind the other, which both cover the same recording area A. The radiation source 3 is configured and the detector unit 4 is designed so that the second detector element 4.2 is not saturated for sections of the recording area A in which the first detector element 4.1 is saturated during irradiation. This can be done, e.g., by advance imaging of the patient P.

In the case of the control device 2, the figures only show the components necessary to explain one or more example embodiments. The radiography system 1 and the associated control devices 2 are essentially known to the person skilled in the art and thus do not need to be explained in detail here.

The control device 2 includes a data acquisition unit 6 that is designed to record a first data set D1 from the first detector element 4.1 and a second data set D2 from the second detector element 4.2, so that both data sets D1, D2 can be separated from one another.

The control device 2 also includes a correction unit 7 designed to correct at least one of the data sets D1, D2. The correction unit 7 is designed, e.g., for noise suppression, pixel registration, correction of cone beam enlargement, and/or spectral correction.

The control device 2 also includes an image generating unit 8 designed to generate a projection image, whose first area, especially a central area, is generated using data from the first data set D1 and especially also using data from the second data set D2, and whose second area, especially a peripheral area, is generated using data from the second data set D2 or data from both data sets D1, D2.

FIG. 2 shows data from imaging according to the prior art. A body area K, e.g. a breast, is irradiated from above with X-rays. The radiation hits the detector unit 4, which here is formed from one individual element, and a signal S is detected by the detector unit, whose path is reproduced in the form of a graph. The detected signal S is low where the body part K is and rises towards the edge. The signal S increases so sharply in the area of the skin-air interface T that the detector unit becomes saturated, which is indicated by a horizontal signal path.

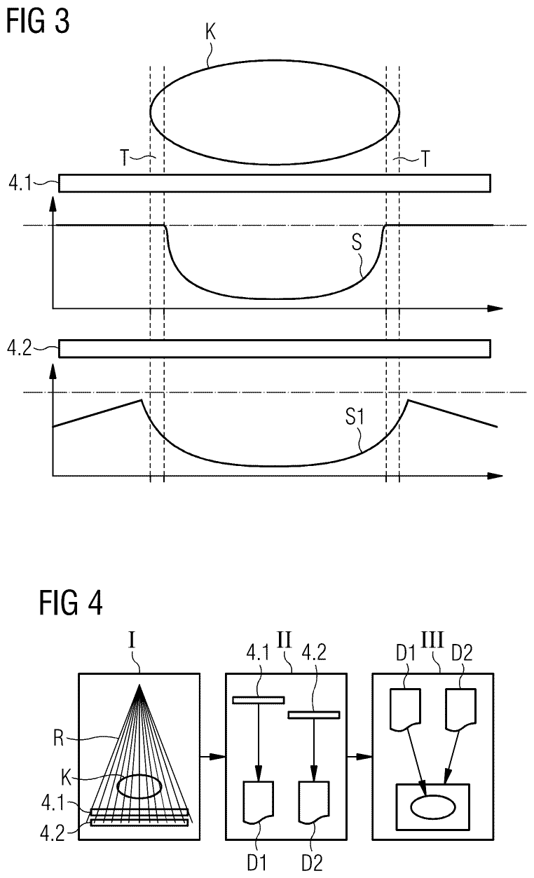

FIG. 3 shows data sets from imaging according to one or more example embodiments; the same body part is imaged here as in FIG. 2. In contrast to FIG. 2, however, a detector unit 4 has now been used for imaging, as shown in FIG. 1, and includes two detector modules 4.1, 4.2. The upper section of the image with the first detector module 4.1 shows as the first signal S the same signal S as FIG. 2, which is also to be expected as the intensity of the radiation is the same.

The lower section of the image with the second detector module 4.2 shows as the second signal S1 a clearly reduced path of intensity, however. It can clearly be seen that the intensity rises sharply in the area of the skin-air interface T, continuing beyond this area and then decreasing.

If we now assume that the two signals S, S1 should show the same path in the area of the body part (more precisely the two inner dotted lines), the second signal S1 can now be scaled so that it shows the same path between the two inner dotted lines. If the other parts of the second signal are scaled accordingly, they show the path the first signal would actually have taken if the detector element had not been saturated.

FIG. 4 shows a method for recording projection images with an imaging system as shown in, e.g., FIG. 1.

Step I involves the irradiation of a body part K of a patient P with the radiation source, wherein both detector elements 4.1, 4.2 of the detector unit 4 are irradiated through the body part K.

Step II involves the reading of both detector elements 4.1, 4.2 and the forming of a first data set D1 by reading the first detector element 4.1 and a second data set D2 by reading the second data element 4.2.

Step III involves the generation of a projection image from both data sets D1, D2, wherein a central area, where the body part was imaged, is formed from the first data set D1 and a peripheral area, in which the first detector element was saturated, is formed from the second data set D2.

Finally, it is noted once again that example embodiments are described, which can be modified by the person skilled in the art in a wide variety of ways without leaving the field of the invention. Moreover, use of the indefinite article “a” or “an” does not prevent the features concerned being present multiple times. Likewise, terms like “unit” do not exclude the possibility that the components concerned comprise multiple interacting subcomponents, which may be distributed, including spatially if applicable. The term “a number” should be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SOC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable information control (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results appropriately achieved by other components or equivalents.

1. An imaging system configured to record projection images, the imaging system comprising:
a detector unit;
a radiation source, wherein
the detector unit includes two detector elements arranged one behind the other, both of the two detector elements cover a same recording area, the two detector elements including a first detector element and a second detector element, and
at least one of the radiation source or the detector unit is configured such that the second detector element is not saturated for sections of the recording area in which the first detector element is saturated during irradiation; and
a data acquisition unit configured to record a first data set of the first detector element and to record a second data set of the second detector element such that first data set and the second data set are separable from one another.

2. The imaging system of claim 1, wherein the radiation source is an X-ray source or a particle radiation source.

3. The imaging system of claim 1, further comprising:
a correction unit configured to correct at least one of the data sets.

4. The imaging system of claim 1, further comprising:
an image generating unit configured to generate a projection image, the projection image having a first area generated using data from the first data set and a second area generated using data from the second data set.

5. The imaging system of claim 1, wherein the second detector element is configured to receive higher energy radiation than the first detector element.

6. The imaging system of claim 1, wherein the first detector element is an absorber.

7. A method for recording projection images with the imaging system of claim 1, the method comprising:
irradiating a body part of a patient with the radiation source;
forming the first data set by reading the first detector element and the second data set by reading the second detector element; and
generating a projection image from the first data set and the second data set, wherein image values in the projection image in which the first detector element was saturated during reading are based on data from the second data set.

8. The method of claim 7, wherein a first area of the projection image that shows the body part is generated from the first data set and a second area of the projection image is generated from the second data set or a combination of the first data set and the second data set.

9. The method of claim 7, wherein the position and signal path on a surface of a recorded subject are extracted from the second data set, and the position and signal path is used to determine a corresponding signal path from the first data set.

10. The method of claim 7, wherein at least one of the first data set or the second data set is corrected with regard to geometric effects related to dual-layer technology.

11. The method of claim 7, wherein the first data set and the second data set are spectrally corrected.

12. A non-transitory computer program product, comprising commands that, when executed by a system, cause the system to perform the method of claim 7.

13. A non-transitory computer-readable storage medium, comprising commands that, when executed by a system, cause the system to perform the method of claim 7.

14. The imaging system of claim 2, wherein the imaging system is a medical technology imaging system.

15. The imaging system of claim 3, wherein the correction unit is configured to perform at least one of:
noise suppression,
pixel registration,
correction of cone beam enlargement, or
spectral correction.

16. The imaging system of claim 4, wherein the first area of the projection image is a central area and the second area of the projection image is a peripheral area.

17. The imaging system of claim 4, the first area of the projection image is generated using the data from the first data set and the data from the second data set.

18. The imaging system of claim 4, wherein image data for which the first detector element was saturated is reconstructed using data from the second data set.

19. The imaging system of claim 6, wherein a detection layer of the first detector or a surface in or on the detection layer is configured such that that the second detector element does not become saturated with a predetermined radiation intensity for image recording.

20. The imaging system of claim 19, wherein the detector unit includes an absorber plate between the first detector element and the second detector element, and the absorber plate at least partially covers the second detector element.
